(19) United States
(12) Patent Application Publication
(10) Pub. No.: US 2024/0000131 A1
(43) Pub. Date: Jan. 4, 2024

(54) CONSUMABLE

(71) Applicant: NICOVENTURES TRADING LIMITED, LONDON (GB)

(72) Inventors: Maria MONTSERRAT SANCHEZ PENA, London (GB); Carla RENTON, London (GB); Martine GELINAS, London (GB)

(21) Appl. No.: 18/005,793

(22) PCT Filed: Jul. 16, 2021
(86) PCT No.: PCT/GB2021/051832

§ 371 Date: Jan. 17, 2023

(30) Foreign Application Priority Data
Jul. 17, 2020 (GB) 2011092.0

(51) Int. Cl. A24B 15/30; A24B 15/167; A24B 15/12; A24B 13/00

(52) U.S. Cl. CPC A24B 15/303; A24B 15/167; A24B 15/12; A24B 13/00

(57) ABSTRACT

The present disclosure relates to a consumable for use in a substance delivery system, the consumable comprising a mint-flavored composition. The present disclosure further relates to a method of manufacturing the consumable, and a use of a botanical to provide a mint aroma.

### PRIORITY CLAIM

The present application is a National Phase entry of PCT Application No. PCT/GB2021/051832, filed Jul. 16, 2021, which claims priority from GB Application No. 2011092.0, filed Jul. 17, 2020, each of which hereby fully incorporated herein by reference.

### TECHNICAL FIELD

The present disclosure relates to a consumable for use in a substance delivery system, the consumable comprising a mint-flavored composition. The present disclosure further relates to a method of manufacturing the consumable, and a use of a botanical to provide a mint aroma.

### BACKGROUND

Flavored compositions are used in various substance delivery systems to enhance a user's experience of the substance being delivered. Aerosol provision systems, for example e-cigarettes, generally contain an aerosolizable material such as a reservoir of a source liquid, typically with a flavoring agent and an optional active ingredient, from which an aerosol is generated for inhalation by a user, e.g. by vaporization. Other source materials may be similarly aerosolized in heating devices, such as a gel or solid material comprising an active ingredient and/or a flavoring agent or flavorant. Flavor can also be introduced in smoking articles such as cigarettes either via the filter element attached to one end of the tobacco rod or via the tobacco material directly. In aerosol-free delivery systems, flavour may be delivered in the form of a pouched oral product such as a smokeless tobacco product, or an oral product containing a tobacco-free substrate.

Irrespective of the product type, however, it is important for flavor to be delivered to the user in an enjoyable form and over a wide spectrum of profiles. In particular, it is desirable if the flavor delivered can be tailored to consumer preference and/or other requirements including any regulatory limitations. For example, it may be preferable to formulate a flavour so as to produce a sensory experience with a particular taste and/or aroma profile.

### SUMMARY

In one aspect the present disclosure provides a consumable for use in a substance delivery system, wherein the consumable comprises a mint-flavored composition comprising: (i) a first botanical or constituent, derivative or extract thereof; (ii) a second, different, botanical or constituent, derivative or extract thereof; wherein the first and second botanicals are selected from members of the Lamiaceae, Geraniaceae, Asteraceae, Myrtaceae or Ericaceae families;

provided that if the first botanical is spearmint or peppermint selected from the group consisting of Mentha x piperita, Mentha viridis, Mentha spicata, Mentha balsamea and Mentha haplocalyx, the second botanical is not spearmint or peppermint selected from the group consisting of Mentha x piperita, Mentha viridis, Mentha spicata, Mentha balsamea and Mentha haplocalyx; wherein the composition has an aroma in addition to mint which is provided by (i) and/or (ii); and the consumable further comprises at least one component selected from aerosol-former materials, active substances, substrate materials, or a combination thereof.

In another aspect, the present disclosure provides a container or package containing the consumable described herein.

In another aspect, the present disclosure provides a substance delivery system comprising the consumable described herein or the container or package described herein.

In another aspect the present disclosure provides a process for preparing a consumable as defined herein, the process comprising: (i) providing a first botanical or constituent, derivative or extract thereof; (ii) providing a second, different botanical or constituent, derivative or extract thereof; wherein the first and second botanicals are selected from members of the Lamiaceae, Geraniaceae, Asteraceae, Myrtaceae or Ericaceae families; provided that if the first botanical is spearmint or peppermint, the second botanical is not spearmint or peppermint; (iii) contacting (i) and (ii) to prepare the mint-flavored composition; (iv) combining the mint-flavored composition with a further component selected from aerosol-former materials, active substances, substrate materials, or a combination thereof, to prepare the consumable.

In another aspect the present disclosure provides the use of a botanical, or constituent, derivative or extract thereof, in a mint-flavored composition to provide an aroma in addition to mint, wherein the botanical, or constituent, derivative or extract thereof, is selected from members of the Geraniaceae, Asteraceae, Myrtaceae, Ericaceae, or Lamiaceae families but it not a member of the Mentha genus, wherein the composition is for use in a substance delivery system.

In another aspect, the present disclosure provides the use of a botanical, or constituent, derivative or extract thereof to provide a mint aroma to a user of a substance delivery system, wherein the botanical or constituent, derivative or extract thereof is a member of a genus other than the Mentha genus.

In another aspect the present disclosure provides the use of a botanical, or constituent, derivative or extract thereof, in a consumable for a substance delivery system to provide an aroma in addition to mint, wherein the botanical, or constituent, derivative or extract thereof is selected from the members of the Lamiaceae, Geraniaceae, Asteraceae, Myrtaceae or Ericaceae families, and the consumable further comprises at least one component selected from aerosol-former materials, active substances, substrate materials, or a combination thereof.

These and other features, aspects, and advantages of the disclosure will be apparent from a reading of the following detailed description together with the accompanying drawings, which are briefly described below. The invention includes any combination of two, three, four, or more of the above-noted features as well as combinations of any two, three, four, or more features or elements set forth in this disclosure, regardless of whether such features or elements are expressly combined in a specific embodiment description herein. This disclosure is intended to be read holistically such that any separable features or elements of the disclosure, in any of its various aspects and embodiments, should be viewed as intended to be combinable unless the context clearly dictates otherwise.

For ease of reference, these and further aspects of the present disclosure are now discussed under appropriate section headings. However, the teachings under each section are not necessarily limited to each particular section.

### BRIEF DESCRIPTION OF THE DRAWING FIGURES

Having thus described aspects of the disclosure in the foregoing general terms, reference will now be made to the accompanying drawings, which are not necessarily drawn to scale. The drawings are exemplary only, and should not be construed as limiting the disclosure.

FIG. 1 is a schematic representation of an electronic aerosol delivery system.

FIG. 2 is a cross-sectional view of a pouched product embodiment, taken across the width of the product, showing an outer pouch filled with a composition of the present disclosure.

### DETAILED DESCRIPTION

In the following description, a number of specific details are presented in order to provide a thorough understanding of the embodiments of the present disclosure. It is, however, to be understood that this disclosure is not limited to these specific details. It is also to be understood that the terminology employed herein is used for the purpose of describing particular embodiments only and is not intended to be limiting since the scope of the present disclosure will be limited only by the appended claims and equivalents thereof. It will also be apparent that specific details known to the person skilled in the art are omitted for the purposes of clarity where appropriate.

As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the composition including water or other solvent. Unless otherwise indicated, reference to “weight percent” (or “% by weight”) of a composition reflects the total wet weight of the composition (i.e., including water or other solvent).

In this specification, unless otherwise stated, the term “about” modifying the quantity of an ingredient refers to variation in the numerical quantity that can occur, for example, through typical measuring and liquid handling procedures used for making concentrates or solutions in the real world; through inadvertent error in these procedures; through differences in the manufacture, source, or purity of the ingredients employed, or to carry out the methods; and the like. The term “about” also encompasses amounts that differ due to different equilibrium conditions for a composition resulting from a particular initial mixture. Whether or not modified by the term “about”, the claims include equivalents to the quantities.

The ranges provided herein provide exemplary amounts of each of the components. Each of these ranges may be taken alone or combined with one or more other component ranges.

### Mint-Flavored Composition

The present disclosure relates to consumable including inter alia a mint-flavored composition where the composition has an aroma in addition to mint that is provided by the Lamiaceae, Geraniaceae, Asteraceae, Myrtaceae or Ericaceae botanicals contained therein.

Flavour is defined by ASTM International (ASTM International, 2009. Standard Terminology Relating to Sensory Evaluations of Materials and Products, E253-09a) as (1) the perception resulting from stimulating a combination of the taste buds, the olfactory organs, and chemesthetic receptors within the oral cavity; or (2) the combined effect of taste sensations, aromatics and chemical feeling factors evoked by a substance in the oral cavity. This definition thus takes into account the three elements of flavor: (1) taste, (2) aromatics or aroma, and (3) trigeminal sensations.

By the term “mint-flavored” is therefore meant the user of the consumable in a substance delivery system experiences a mint (or minty) taste, smell and feeling from the composition. A mint flavour is, for example, typically described as “fresh”, “cool”, “cold”, “refreshing” and/or “clean”; it can be described as a combination of a sweet taste, a fresh, aromatic aroma and a cooling aftertaste or sensation. A “mint-flavor” or “minty flavor” would be readily identified by the person skilled in the art.

Aroma refers to a scent or smell and in the present disclosure is intended to mean the orthonasal smell of an object, i.e. the perception of molecules that enter directly through the nose and up the nasal passages. By the expression “aroma in addition to mint” is therefore meant a scent or small other than mint, it being appreciated that the mint flavour of the composition will give rise to a mint aroma/scent. The identity of the additional aroma is not limited and can include any scent known in the art. Exemplary additional aromas include: apple, pineapple, chocolate, berry (e.g. raspberry, blueberry, strawberry), grapefruit, ginger, banana, orange, lemon, lime, pear, mango, basil, cinnamon, coffee, lavender, oregano, wintergreen, coconut, melon, blackcurrant, grape, anise, rose, licorice, root beer, coconut, grape, green pepper, hazelnut, marshmallow, peach, thyme, basil, oregano, bay leaf and bergamot. As the person skilled in the art will appreciate, the additional aroma may be a combination of scents, with one scent being more dominant than another. The present disclosure is not limited in this respect; all that is required is for the additional aroma to be identifiable.

In various embodiments of the present disclosure, the additional aroma is selected from the group consisting of: citrus, fruity, herbal, floral, creamy (e.g. chocolate), roasted, nutty or a combination thereof. In various embodiments of the present disclosure, the additional aroma is selected from the group consisting of: lemon, bergamot, orange, floral, grapefruit, lime, banana, pineapple, apple, pear, mango, basil, cinnamon, chocolate, strawberry, raspberry, ginger, lavender, coconut, melon, blackcurrant, grape, anise, rose, liquorice, root beer, coconut, grape, green pepper, hazelnut, marshmallow, peach, thyme, basil, oregano, bay leaf, or a combination thereof.

In various embodiments of the present disclosure the additional aroma is selected from the group consisting of: apple, floral, pineapple, chocolate, berry (e.g. raspberry, blueberry, strawberry), grapefruit, ginger, banana, orange, lemon, lime, pear, mango, basil, cinnamon, coffee, lavender, oregano, wintergreen and bergamot, or a mixture thereof. For example the additional aroma may be selected from the group consisting of: lemon, bergamot, orange, floral, grapefruit, lime, banana, pineapple, apple, pear, mango, basil, cinnamon, chocolate, strawberry, raspberry, ginger, lavender, or a mixture thereof.

The presence of an additional aroma can be determined through user testing and perception, laboratory techniques and/or with headspace technology. Headspace technology is a technique to elucidate the odor or aroma compounds present in the air surrounding an object of interest. The equipment to carry out the technique is readily available and involves a hollow dome or sphere-like object, which forms an airtight seal and surrounds the object of interest.

Inert gases are passed into the space containing the object or a vacuum is established such that the odor or aroma compounds are removed from the headspace. These compounds are in turn captured using a variety of techniques, among them cold surfaces, solvent traps, and adsorbent materials. Once captured, the samples can be analyzed using techniques such as gas chromatography, mass spectrometry or carbon-13 NMR and the elucidated compounds compared against known libraries of aroma compounds to identify the scent being produced.

Laboratory techniques that may be used include solid-phase microextraction, dynamic headspace extraction or stir bar sorption extraction. Typically, solid-phase microextraction or SPME may be used. SPME is a solid phase extraction sampling technique that involves the use of a fiber coated with an extracting phase, that can be a liquid (e.g. polymer) or a solid (sorbent) which extracts different analytes (including both volatile and non-volatile) from different kinds of media, that can be in liquid or gas phase. After extraction, the SPME fiber is transferred to the injection port of separating instruments such as a gas chromatography and mass spectrometry for analysis. There are many commercially available SPME fiber coatings.

The present disclosure requires the aroma in addition to mint to be provided by the first botanical and/or the second botanical, where the botanicals are selected from members of the Lamiaceae, Geraniaceae, Asteraceae, Myrtaceae or Ericaceae families. This means that whilst the composition is mint-flavored, the user experiences a broader spectrum of scent. Through the additional aroma, the mint-flavored composition of the present disclosure thus provides a “non-traditional mint experience” and can be described as a “non-traditional mint”.

Unlike the currently available mint flavors for substance delivery systems such as e-cigarettes, tobacco heating devices, smoking articles or oral products, the composition of the present disclosure provides a unique sensation by delivering a multi-sensorial experience. Not only is the user provided with the desired mint flavor, but they receive a further sensation from the additional aroma. The additional non-mint aroma can, for instance, be chosen to stimulate a desired sensation in a user, for example: a refreshing taste of grapefruit, warm notes of ginger, floral lavender notes, hints of lemon and lime zest, sweet citrus etc. It allows the manufacturer to enhance the user experience and broaden the “mint” products available to consumers.

The form of the mint-flavored composition is not limited and will typically depend on the substance delivery system in which it is intended to be used. The composition may be a liquid, solid or gel. The composition may, for example, be a powder, dust, crushed particles, granules, pellets, shreds, strips, sheets, or the like. In various embodiments of the present disclosure, the composition is in the form of a liquid, solid or gel. In various embodiments of the present disclosure, the composition is in the form of a liquid or gel.

The mint-flavored composition may also be loaded onto a substrate material within the consumable. The substrate or support may, for example, be or comprise a carrier material such as paper, card, paperboard, cardboard, tobacco, reconstituted material (e.g. reconstituted tobacco), a plastics material, a ceramic material, a composite material, a polymeric (e.g. fibrous) material, glass, a metal, or a metal alloy. Substrate materials are discussed in more detail below.

### Botanicals

The composition of the present disclosure comprises one or more constituents, derivatives or extracts of a first botanical and one or more constituents, derivatives or extracts of a second botanical. The first and second botanicals are different from one another and are selected from the members of the Lamiaceae, Geraniaceae, Asteraceae, Myrtaceae or Ericaceae families. There is also a proviso that if the first botanical is spearmint or peppermint, the second botanical is not spearmint or peppermint. By the terms “spearmint or peppermint” is meant the specific species and hybrid: Mentha x piperita, Mentha viridis, Mentha spicata, Mentha balsamea and Mentha haplocalyx, it does not extend to other types or varieties of peppermint and spearmint. The additional aroma of the composition is thereby provided by a botanical, constituent, derivative or extract thereof other than “traditional” mint.

As used herein, the term “botanical” includes any material derived from a plant including, but not limited to, extracts, leaves, bark, fibers, stems, roots, seeds, flowers, fruits, pollen, husk, shells or the like. The material may be in the form of liquid, gas, solid, gel, powder, dust, crushed particles, granules, pellets, shreds, strips, sheets, or the like. In various embodiments of the present disclosure the material is in the form of liquid, solid, gel, powder, crushed particles, granules, pellets, shreds, strips, sheets or the like.

The Lamiaceae family are a family of flowering plants commonly known as the mint family. They may also be known as the Labiatae family. Many of the plants are aromatic in all parts and include widely used herbs such as basil, mentha, rosemary, sage, savory, marjoram, oregano, hyssop, thyme and lavender. At the time of writing, the last revision of the family was published in 2004 by Harley et al., “Labiatae” pages 167-275 of The Families and Genera of Vascular Plants volume VII. Springer-Verlag: Berlin; Heidelberg, Germany. This revision is incorporated herein by reference; it described and provided keys to 236 genera. A list at the Angiosperm Phylogeny website is also frequently updated: http://www.mobot.org/mobot/research/apweb/genera/lamiaceaegen.html. This list is incorporated herein by reference.

In various embodiments of the present disclosure, the Lamiaceae botanical is selected from members of the following genera: Acanthomintha, Achyrospermum, Acinos, Acrocephalus, Acrotome, Acrymia, Adelosa, Aegiphila, Aeollanthus, Agastache, Ajuga, Ajugoides, Alajja, Alvesia, Amasonia, Amethystea, Anisochilus, Anisomeles, Asterohyptis, Ballota, Basilicum, Becium, Benguellia, Blephilia, Bostrychanthera, Bovonia, Brachysola, Brazoria, Bystropogon, Calamintha, Callicarpa, Capitanopsis, Capitanya, Caryopteris, Catoferia, Cedronella, Ceratanthus, Chaiturus, Chamaesphacos, Chaunostoma, Chelonopsis, Chloanthes, Cleonia, Clerodendrum, Clinopodium, Colebrookea, Collinsonia, Colquhounia, Comanthosphace, Congea, Conradina, Coridothymus, Cornutia, Craniotome, Cryphia, Cuminia, Cunila, Cyanostegia, Cyclotrichium, Cymaria, Dauphinea, Dicerandra, Dicrastylis, Discretitheca, Dorystoechas, Dracocephalum, Drepanocaryum, Elsholtzia, Endostemon, Englerastrum, Eremostachys, Eriope, Eriophyton, Eriopidion, Eriothymus, Erythrochlamys, Euhesperida, Eurysolen, Faradaya, Fuerstia, Galeopsis, Garrettia, Geniosporum, Glechoma, Glechon, Glossocarya, Gmelina, Gomphostemma, Gontscharovia, Hanceola, Haplostachys, Haumaniastrum, Hedeoma, Hemiandra, Hemigenia, Hemiphora, Hemizygia, Hesperozygis, Heterolamium, Hoehnea, Holmskioldia, Holocheila, Holostylon, Horminum, Hosea, Hoslundia, Huxleya, Hymenocrater, Hymenopyramis, Hypenia, Hypogomphia, Hyptidendron, Hyptis, Hyssopus, lsodictyophorus, lsodon, lsoleucas, Kalaharia, Karomia, Keiskea, Killickia, Kudrjaschevia, Kurzamra, Lachnostachys, Lagochilus, Lagopsis, Lallemantia, Lamiophlomis, Lamium, Lavandula, Leocus, Leonotis, Leonurus, Lepechinia, Leucas, Leucophae, Leucosceptrum, Limniboza, Lophanthus, Loxoca/yx, Lycopus, Macbridea, Madlabium, Marmoritis, Marrubium, Marsypianthes, Matsumurella, Meehania, Melissa, Melittis, Mentha, Meriandra, Mesona, Metastachydium, Microcorys, Micromeria, Microtoena, Minthostachys, Moluccella, Monarda, Monardella, Monochilus, Mosla, Neoeplingia, Neohyptis, Neorapinia, Nepeta, Newcastelia, Nosema, Notochaete, Obtegomeria, Ocimum, Octomeron, Ombrocharis, Oncinocalyx, Origanum, Orthosiphon, Otostegia, Ovieda, Oxera, Panzerina, Paralamium, Paraphlomis, Paravitex, Peltodon, Pentapleura, Perilla, Perillula, Peronema, Perovskia, Perrierastrum, Petitia, Petraeovitex, Phlomidoschema, Phlomis, Phlomoides, Phyllostegia, Physopsis, Physostegia, Piloblephis, Pitardia, Pityrodia, Platostoma, Plectranthus, Pogogyne, Pogostemon, Poliomintha, Prasium, Premna, Prostanthera, PruneIla, Pseuderemostachys, Pseudocarpidium, Pseudocaryopteris, Pseudomarrubium, Puntia, Pycnanthemum, Pycnostachys, Rabdosiella, Renschia, Rhabdocaulon, Rhaphiodon, Rhododon, Rosmarinus, Rostrinucula, Rotheca, Roylea, Rubiteucris, Rydingia, Sabaudia, Saccocalyx, Salazaria, Salvia, Satureja, Schizonepeta, Schnabelia, Scutellaria, Sideritis, Siphocranion, Solenostemon, Spartothamnella, Sphenodesme, Stachydeoma, Stachyopsis, Stachys, Stenogyne, Sulaimania, Suzukia, Symphorema, Symphostemon, Synandra, Syncolostemon, Tectona, Teijsmanniodendron, Tetraclea, Tetradenia, Teucridium, Teucrium, Thomcroftia, Thuspeinanta, Thymbra, Thymus, Tinnea, Trichostema, Tripora, Tsoongia, Vitex, Viticipremna, Volkameria, Wamockia, Wenchengia, Westringia, Wiedemannia, Wrixonia, Xenopoma, Zataria, Zhumeria, and Ziziphora.

In various embodiments of the present disclosure at least one of the first and second botanicals, or constituent, derivative or extract thereof, is a member of the Lamiaceae family. In various embodiments of the present disclosure both of the first and second botanicals, or constituent, derivative or extract thereof are members of the Lamiaceae family.

In various embodiments of the present disclosure at least one of the first and second botanicals, or constituent, derivative or extract thereof, is a member of the Menthae tribe. In various embodiments both of the first and second botanicals, or constituent, derivative or extract thereof, are selected from members of the Menthae tribe. The Menthae tribe is part of the Lamiaceae family and includes members of the following genera from the above list: Lycopus, Acanthomintha, Blephilia, Bystropogon, Clinopodium, Conradina, Cuminia, Cunila, Cyclotrichium, Dicerandra, Drymosiphon, Eriothymus, Glechon, Gontscharovia, Hedeoma, Hesperozygis, Hoehnea, Killickia (treated as part of Micromeria by Harley et al. 2004), Kurzamra, Mentha, Micromeria, Minthostachys, Monarda, Monardella, Obtegomeria, Origanum, Pentapleura, Piloblephis, Pogogyne, Poliomintha, Pycnanthemum, Rhabdocaulon, Rhododon, Saccocalyx, Satureja, Stachydeoma, Thymbra, Thymus, Zataria, Ziziphora, Agastache, Cedronella, Dracocephalum, Drepanocaryum, Glechoma, Heterolamium, Hymenocrater, Hyssopus, Kudrjaschevia, Lallemantia, Lophanthus, Marmoritis, Meehania, Nepeta, Schizonepeta, Cleonia, Horminum, PruneIla, Chaunostoma, Dorystaechas, Lepechinia, Melissa, Meriandra, Neoeplingia, Perovskia, Rosmarinus, Salvia, Zhumeria, Acinos (treated as part of Clinopodium by Harley et al. 2004), Calamintha, and Pitardia.

In various embodiments of the present disclosure at least one of the first and second botanicals, or constituent, derivative or extract thereof, is selected from members of the: Agastache, Mentha, Monarda, Minthostachys or Melissa genera. In various embodiments of the present disclosure both of the first and second botanicals, or constituent, derivative or extract thereof are members of the: Agastache, Mentha, Monarda, Minthostachys or Melissa genera.

In various embodiments of the present disclosure the first botanical or constituent, derivative or extract thereof is a member of a genus selected from the group consisting of: Agastache, Mentha, Monarda, Minthostachys and Melissa, and the second botanical or constituent, derivative or extract thereof is a member of the Mentha genus. In various embodiments of the present disclosure, at least one of the first and second botanicals, or constituent, derivative or extract thereof, is a member of the Mentha genus. In various embodiments, both of the first and second botanicals, or constituent, derivative or extract thereof, are selected from members of the Mentha genus.

Agastache is a genus of aromatic flowing herbaceous perennial plants which includes 22 species. As a whole, the genus is known as giant hyssops or hummingbird mints. The sections with their species are: Agastache sect. Agastache [Agastache cusickii, Agastache foeniculum, Agastache nepetoides, Agastache occidentalis, Agastache parvifolia, Agastache rugose, Agastache scrophulariifolia, Agastache urticifolia] and Agastache sect. Brittonastrum: [Agastache aurantiaca, Agastache breviflora, Agastache cana, Agastache coccinea, Agastache eplingiana, Agastache meamsii, Agastache Mexicana, Agastache micrantha, Agastache pallida, Agastache pallidiflora, Agastache palmeri, Agastache pringlei, Agastache rupestris, Agastache wrightii]. In various embodiments of the present disclosure the Agastache botanical, or constituent, derivative or extract thereof, is a member of the Agastache rugose or Agastache rupestris species, e.g. Agastache rugose (Korean mint).

Throughout this disclosure the use of the term “species” and any specifically named species includes all subspecies, varieties, subvarieties, forms and subforms, along with any hybrids and/or cultivars thereof.

The Monarda genus includes the following species: Monarda bartletti, Monarda bradburiana, Monarda citriodora, Monarda clinopodia, Monarda clinopodioides, Monarda didyma, Monarda eplingiana, Monarda fistulosa, Monarda fruticulosa, Monarda humilis, Monarda lindheimeri, Monarda luteola, Monarda maritima, Monarda media, Monarda x medioides, Monarda pectinate, Monarda pringlei, Monarda punctate, Monarda russeliana, Monarda stanfieldii, Monarda viridissima. In various embodiments of the present disclosure the Monarda botanical, or constituent, derivative or extract thereof, is a member of the Monarda citriodora or Mondara didyma species, e.g. Monarda citriodora (lemon mint).

Melissa is a genus of perennial herbs and the most commonly grown species of this genus is Melissa officinalis, commonly known as lemon balm or balm. Other species include: Melissa axillaris Melissa flava and Melissa yunnanensis. In various embodiments of the present disclosure, the Melissa botanical or constituent, derivative or extract thereof is a member of the Melissa officinalis (lemon balm) species.

Mentha is a genus in which the distinction between species is more complex; it is estimated that 13 to 24 species exist because hybridization occurs naturally and some species overlap. As is known in the art, plant hybrids are generally given names that include an “x”, e.g. Mentha x piperita. Many cultivars also exist within the Mentha genus—an assemblage of plants selected for desirable characteristics that are maintained during propagation. Every unique cultivar has a unique name which is regulated by the International Code of Nomenclature for Cultivated Plants; this name consists of a botanical name followed by a cultivar epithet, e.g. Mentha spicata ‘Russian’. As noted above, the present disclosure includes hybrids and cultivars.

The botanical or constituent, derivative or extract thereof which is a member of the Mentha genus may therefore be a Mentha species, Mentha hybrid or Mentha cultivar. In various embodiments of the present disclosure, the botanical or constituent, derivative or extract thereof which is a member of the Mentha genus is selected from the following species: Mentha alaica, Mentha aquatica, Mentha arvensis, Mentha atrolilacina, Mentha australis, Mentha balsamea, Mentha canadensis, Mentha cervina, Mentha cunninghamii, Mentha dahurica, Mentha darvasica, Mentha diemenica, Mentha gattefossei, Mentha grandiflora, Mentha haplocalyx, Mentha japonica, Mentha laxiflora, Mentha longifolia, Mentha micrantha, Mentha nemorosa, Mentha niliaca, Mentha pamiroalaica, Mentha pulegium, Mentha requienii, Mentha royleana, Mentha sachalinensis, Mentha satureioides, Mentha spicata, Mentha suaveolens, Mentha aquatica brevis, Mentha viridis, or a hybrid and/or cultivar thereof.

In various embodiments the Mentha botanical or constituent, derivative or extract thereof is selected from the following species: Mentha alaica, Mentha aquatica, Mentha arvensis, Mentha atrolilacina, Mentha australis, Mentha canadensis, Mentha cervina, Mentha cunninghamii, Mentha dahurica, Mentha darvasica, Mentha diemenica, Mentha gattefossei, Mentha grandiflora, Mentha japonica, Mentha laxiflora, Mentha longifolia, Mentha micrantha, Mentha nemorosa, Mentha niliaca, Mentha pamiroalaica, Mentha pulegium, Mentha requienii, Mentha royleana, Mentha sachalinensis, Mentha satureioides, Mentha suaveolens, Mentha aquatica brevis, or a hybrid and/or cultivar thereof.

Recognized hybrids of the Mentha genus include:

Mentha x carinthiaca (M. arvensis x M. suaveolens), Mentha x dalmatica (M. arvensis x M. longifolia), Mentha x dumetorum (M. aquatica x M. longifolia), Mentha x gayeri, Mentha x gentilis, (syn. Mentha x gracilis; M. arvensis x M. spicata), Mentha x kuemmerlei, Mentha x locyana, Mentha x piperita (M. aquatica x M. spicata), Mentha x pyramidalis, Mentha x rotundifolia (M. longifolia x M. suaveolens), Mentha x suavis, (syn. Mentha x maximilianea; M. aquatica x M. suaveolens), Mentha x verticillata (M. aquatica x M. arvensis), Mentha x villosa (M. spicata x M. suaveolens, also called M. nemorosa), Mentha x villosa-nervata, (M. longifolia x M. spicata), Mentha x wirtgeniana (syn. Mentha x smithiana; M. aquatica x M. arvensis x M. spicata), and cultivars thereof.

Cultivars within the Mentha genus are numerous and include e.g. Mentha suaveolens Variegata, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata ‘Chocolate’, Mentha x piperita ‘Lavender’, Mentha x gracilis ‘Variegata’, Mentha arvensis ‘Banana’, Mentha spicata var. cripsa ‘Moroccan’, Mentha spicata var. crispa ‘Persian’, Mentha spicata ‘Russian’, Mentha piperita ‘Canyon’, Mentha ‘wild berries’, Mentha ‘sweet pear’, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, and Mentha ‘Sweet Bay’.

The present disclosure is not, however, limited to these species, hybrids or cultivars or in fact the Mentha genus. In various embodiments of the present disclosure at least one of the first and second botanicals, or constituent, derivative or extract thereof, is selected from members of the Geraniaceae, Asteraceae, Myrtaceae, Ericaceae or Lamiaceae families but is not a member of the Mentha genus.

The members of the Lamiaceae family other than members of the Mentha genus are discussed above. This discussion is generally applicable, and includes for example, Agastache (e.g. Agastache rugose, Agastache rupestris ‘Apache Sunset’), Calamintha (e.g. Calamintha Nepeta), Pycnanthemum (e.g. Pycnanthemum pilosum), Micromeria (e.g. Micromeria fruticose), Salvia (e.g. Salvia elegans, Salvia elegans ‘Tangerine’, Salvia dorisiana, Salvia elegans ‘Honey Melon’, Salvia discolor, Salvia melissodora), Ocimum (e.g. Ocimum basilicum ‘cinnamon’, Ocimum basilicum ‘anise’ Ocimum selloi) or Thymus (e.g. Thymus sp., Thymus ‘TM122’, Thymus sp).

In various embodiments of the present disclosure, at least one of the first and second botanicals, or constituent, derivative or extract thereof, is selected from members of the Geraniaceae family. Geraniaceae is a family of flowering plants placed in the order Geraniales. The family comprises 830 species in five to seven genera, the largest genera are Geranium, Pelargonium and Erodium. In various embodiments, the Geraniaceae botanical or constituent, derivative or extract thereof is selected from members of the Pelargonium genus. The Pelargonium genus has between 200 and 280 species. At the time of writing, the Plant List accepted 250 species at http://www.theplantlist.org/1.1/browse/A/Geraniaceae/Pelamonium/—this list is incorporated herein by reference.

In various embodiments of the present disclosure the Geraniaceae botanical or constituent, derivative or extract thereof is selected from Pelargonium species, hybrids or cultivars. Cultivated pelargoniums are commonly divided into six groups: A) Zonal, B) Ivy-leaved, C) Regal, D) Angel, E) Unique, F) Scented-leaved, G) Species, and H) Primary hybrids. In various embodiments of the present disclosure the Pelargonium botanical or constituent, derivative or extract thereof is selected from members of group F. Group F are perennials grown chiefly for their scent and include:
Almond—Pelargonium quercifolium; Apple—Pelargonium odoratissimum;
Apple—Pelargonium cordifolium; Apple/Mint—Pelargonium album;
Apricot/Lemon—Pelargonium scabrum; Balsam—Pelargonium panduriforme;
Camphor—Pelargonium botulinum; Celery—Pelargonium ionidiflorum;
Cinnamon—Pelargonium ‘Ardwyck Cinnamon’; Coconut—Pelargonium grossalarioides (Pelargonium parriflorum); Eau de Cologne—Pelargonium ‘Brilliantine’;
Eucalyptus—Pelargonium ‘Secret Love’; Grapefruit—Pelargonium ‘Poquita’;
Ginger—Pelargonium ‘Torrento’ or ‘Cola Bottles’ which is a variety of Pelargonium x nervosum;
Hazelnut—Pelargonium ‘Odorata Hazelnut’; Lavender—Pelargonium ‘Lavender Lindy’; Lemon Pelargonium crispum; Lemon—Pelargonium citronellum (Synonym—Pelargonium ‘Mabel Grey’); Lemon Balm—Pelargonium x melissinum;
Lime—Pelargonium x nervosum; Myrrh—Pelargonium myrrhifolium; Nutmeg—Pelargonium x fragrans; Old Spice—Variety of Pelargonium x fragrans; Orange—Pelargonium x citriodorum (Synonym—Pelargonium ‘Prince of Orange’); Peach—Pelargonium ‘Peaches and Cream’;
Peppermint—Pelargonium tomentosum; Pine—Pelargonium denticulatum;
Pineapple—Pelargonium ‘Brilliant’; Raspberry—Pelargonium ‘Red Raspberry’;
Rose—Pelargonium graveolens (Synonym—Pelargonium roseum); Rose—Pelargonium capitatum; Rose—Pelargonium radens; Southernwood—Pelargonium abrotanifolium;
Spicy—Pelargonium exstipulatum; Strawberry—Pelargonium x scarboroviae; and the cultivars: ‘Attar of Roses’—a cultivar of P. capitatum; ‘Crowfoot Rose’—a cultivar of P. radens; ‘Dr. Livingston’—a cultivar of P. radens; ‘Grey Lady Plymouth’—a cultivar of P. graveolens; and ‘Prince Rupert’—a cultivar of P. crispum.

In various embodiments of the present disclosure, the Pelargonium botanical or constituent, derivative or extract thereof is selected from Perlargonium ‘Pungent Peppermint’, and Pelargonium x scarboroviae.

In various embodiments of the present disclosure, at least one of the botanicals, or constituent, derivative or extract thereof, is selected from members of the Asteraceae family. Asteraceae (otherwise known as Compositae) is a large family of flowering plants. The family includes over 32,000 currently accepted species in over 1,900 genera in 13 subfamilies. In various embodiments of the present disclosure the Asterceae botanical, or constituent, derivative or extract thereof, is selected from members of the Asteroideae subfamily. The Asteroideae subfamily is made up of several tribes including Astereae, Calenduleae, Eupatorieae, Gnaphalieae, Heliantheae, Senecioneae and Tageteae; these tribes are grouped into three supertribes: Senecionodae, Asterodae and Helianthodae. In various embodiments of the present disclosure, the Asterceae botanical, or constituent, derivative or extract thereof, is selected from members of the Helianthodae supertribe of the Asteroideae subfamily, e.g. the Tageteae tribe.

The Tageteae tribe consists of approximately 216 species divided among 28 genera including: Adenophyllum, Bajacalia, Boeberastrum, Boeberoides, Chrysactinia, Clappia, Comaclinium, Coulterella, Dysodiopsis, Dyssodia, Gymnolaena, Haploesthes, Harnackia, Hydropectis, Lescaillea, Leucactinia, Jamesianthus, Jaumea, Nicolletia, Oxypappus, Pectis, Porophyllum, Pseudoc/appia, Sartwellia, Schizotrichia, Strotheria, Tagetes, Thymophylla, Urbinella, and Varilla. In various embodiments of the present disclosure the Asterceae botanical, or constituent, derivative or extract thereof is selected from members of the Tagetes genera of the Tageteae family, for example, Tagetes lemmonii.

In various embodiments of the present disclosure, at least one of the botanicals, or constituent, derivative or extract thereof, is selected from members of the Myrtaceae family. Myrtaceae is a family of dicotyledonous plants placed within the order Myrtales. Myrtle, pohutukawa, bay rum tree, clove, guava, acca (feijoa), allspice, and eucalyptus are some notable members of this group. All species are woody and contain essential oils. In various embodiments of the present disclosure the Myrtaceae botanical, or constituent, derivative or extract thereof, is selected from members of the subfamily Myrtoideae, e.g. Myrtae or Eucalypteae.

In various embodiments of the present disclosure at least one of the botanicals, or constituent, derivative or extract thereof, is selected from members of Eucalypteae, for example members of a genera selected from: Allosyncarpia, Angophora, Arillastrum, Corymbia, Eucalyptopsis, Eucalyptus, and Stockwellia. In various embodiments of the present disclosure at least one of the botanicals, or constituent, derivative or extract thereof, is selected from members of the Eucalyptus genus, for example, Eucalyptus radiata.

In various embodiments of the present disclosure, at least one of the botanicals, or constituent, derivative or extract thereof, is selected from members of the Ericaceae family. The Ericaceae family includes 9 subfamilies: Enkianthoideae (one genus, 16 species), Pyroloideae (4 genera, species), Monotropoideae (10 genera, 15 species), Arbutoideae (up to six genera, about species), Cassiopoideae (one genus, 12 species), Ericoideae (19 genera, 1790 species), Harrimanelloideae (one genus, two species), Styphelioideae (35 genera, 545 species), and Vaccinioideae (50 genera, 1580 species). In various embodiments of the present disclosure at least one of the botanicals, or constituent, derivative or extract thereof, is selected from members of the Vaccinioideae subfamily of Ericaceae; this family contains e.g. cranberry, blueberry, bilberry, lingonberry and huckleberry.

One of the genera of the Vaccinioideae subfamily is Gaultheria; this includes about 135 species including American wintergreen or Gaultheria procumbens. In various embodiments of the present disclosure at least one of the botanicals, or constituent, derivative or extract thereof, is selected from members of the genus Gaultheria, e.g. Gaultheria procumbens.

As noted above, the present disclosure prescribes that if the first botanical is a species selected from the group consisting of: Mentha x piperita, Mentha viridis, Mentha spicata, Mentha balsamea and Mentha haplocalyx, the second botanical is not a species selected from the group consisting of: Mentha x piperita, Mentha viridis, Mentha spicata, Mentha balsamea and Mentha haplocalyx. The purpose of this disclaimer is to exclude traditional mint sources—namely spearmint and peppermint from the Mentha genus—providing the mint flavor and additional aroma. The present disclosure provides mint flavor and an additional aroma without reliance on Mentha-derived spearmint and peppermint. This enables the spectrum of “mint” flavors to be much wider than previously recognized.

In various embodiments of the present disclosure, at least one of the first and second botanicals, or constituent, derivative or extract thereof, is independently selected from the group consisting of: Mentha x piperita, Mentha viridis, Mentha spicata, Mentha balsamea, Mentha haplocalyx, Mentha suaveolens, Mentha suaveolens Variegata, Melissa officinalis, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata, Monarda didyma, Mentha x piperita f. citrata ‘Chocolate’, Mentha x piperita ‘Lavender’, Mentha x gracilis, Mentha x gracilis ‘Variegata’, Mentha arvensis ‘Banana’, Mentha aquatica, Mentha x villosa, Mentha nemorosa, Mentha spicata var. cripsa ‘Moroccan’, Mentha sachalinensis, Agastache rugose, Mentha spicata var. crispa ‘Persian’, Mentha spicata ‘Russian’, Mentha requienii, Mentha arvensis, Mentha arvensis L. var. canandensis, Mentha canadensis, Mentha niliaca, calamintha Nepeta, Mentha australis, Mentha piperita ‘Canyon’, Mentha ‘wild berries’, Mentha ‘sweet pear’, Mentha laxiflora, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’, Pycnanthemum pilosum, Micromeria fruticose, Perlargonium ‘Pungent Peppermint’, Pelargonium x scarboroviae, Agastache rupestris ‘Apache Sunset’, Salvia elegans, Salvia elegans ‘Tangerine’, Eucalyptus radiata, Tagetes lemmonii, Salvia dorisiana, Salvia elegans ‘Honey Melon’, Salvia discolor, Salvia melissodora, Ocimum basilicum ‘cinnamon’, Ocimum basilicum ‘anise’, Ocimum selloi, Thymus sp., Thymus ‘TM122’, Thymus sp., Gaultheria procumbens, or hybrids or cultivars thereof.

In various embodiments of the present disclosure, at least one of the first and second botanicals, or constituent, derivative or extract thereof, is independently selected from the group consisting of: Mentha x piperita, Mentha viridis, Mentha spicata, Mentha balsamea, Mentha haplocalyx, Mentha suaveolens, Melissa officinalis, Mentha x piperita f. citrata, Mentha spicata subsp. citrata, Monarda didyma, Mentha x gracilis, Mentha x gracilis, Mentha arvensis, Mentha aquatica, Mentha x villosa, Mentha nemorosa, Mentha spicata var. cripsa, Mentha sachalinensis, Agastache rugose, Mentha persian, Mentha requienii, Mentha arvensis L. var. canadensis, Mentha canadensis, Mentha niliaca, Calamintha nepeta, Mentha australis, Mentha laxiflora, hybrids and/or cultivars thereof, or a combination thereof.

In various embodiments of the present disclosure, each of the first and second botanicals, or constituent, derivative or extract thereof, is independently selected from the group consisting of: Mentha x piperita, Mentha viridis, Mentha spicata, Mentha balsamea, Mentha haplocalyx, Mentha suaveolens, Mentha suaveolens Variegata, Melissa officinalis, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata, Monarda didyma, Mentha x piperita f. citrata ‘Chocolate’, Mentha x piperita ‘Lavender’, Mentha x gracilis, Mentha x gracilis ‘Variegata’, Mentha arvensis ‘Banana’, Mentha aquatica, Mentha x villosa, Mentha nemorosa, Mentha spicata var. cripsa ‘Moroccan’, Mentha sachalinensis, Agastache rugose, Mentha spicata var. crispa ‘Persian’, Mentha spicata ‘Russian’, Mentha requienii, Mentha arvensis, Mentha arvensis L. var. canadensis, Mentha canadensis, Mentha niliaca, Calamintha nepeta, Mentha australis, Mentha piperita ‘Canyon’, Mentha ‘wild berries’, Mentha ‘sweet pear’, Mentha laxiflora, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’, Pycnanthemum pilosum, Micromeria fruticose, Perlargonium ‘Pungent Peppermint’, Pelargonium x scarboroviae, Agastache rupestris ‘ Apache Sunset’, Salvia elegans, Salvia elegans ‘Tangerine’, Eucalyptus radiata, Tagetes lemmonii Salvia dorisiana, Salvia elegans ‘Honey Melon’, Salvia discolor, Salvia melissodora, Ocimum basilicum ‘cinnamon’, Ocimum basilicum ‘anise’, Ocimum selloi, Thymus sp., Thymus ‘TM122’, Thymus sp., Gaultheria procumbens, or hybrids or cultivars thereof.

In various embodiments of the present disclosure, each of the first and second botanicals, or constituent, derivative or extract thereof, is independently selected from the group consisting of: Mentha x piperita, Mentha viridis, Mentha spicata, Mentha balsamea, Mentha haplocalyx, Mentha suaveolens, Melissa officinalis, Mentha x piperita f. citrata, Mentha spicata subsp. citrata, Monarda didyma, Mentha x gracilis, Mentha x gracilis, Mentha arvensis, Mentha aquatica, Mentha x villosa, Mentha nemorosa, Mentha spicata var. cripsa, Mentha sachalinensis, Agastache rugose, Mentha requienii, Mentha arvensis L. var. canadensis, Mentha canadensis, Mentha niliaca, Calamintha nepeta, Mentha australis, Mentha laxiflora, hybrids and/or cultivars thereof, or a mixture thereof.

The cultivars of the above-mentioned species are not limited and can, for example include: Mentha spicata var. crispa ‘Persian’, Mentha suaveolens ‘Variegata’, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata ‘Chocolate’, Mentha x piperita ‘Lavender’, Mentha x gracilis ‘Variegata’, Mentha arvensis ‘Banana’, Mentha spicata var. cripsa ‘Moroccan’, Mentha spicata ‘Russian’, Mentha piperita ‘Canyon’, Mentha ‘wild berries’, Mentha ‘sweet pear’, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’ or a combination thereof.

In various embodiments of the present disclosure, at least one of the first and second botanicals, or constituent, derivative or extract thereof, is selected from the group consisting of: Mentha x piperita, Mentha viridis, Mentha spicata, Mentha balsamea, Mentha haplocalyx, Mentha suaveolens, Mentha suaveolens Variegata, Melissa officinalis, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata, Monarda didyma, Mentha x piperita f. citrata ‘Chocolate’, Mentha x piperita ‘Lavender’, Mentha x gracilis, Mentha x gracilis ‘Variegata’, Mentha arvensis ‘Banana’, Mentha aquatica, Mentha x villosa, Mentha nemorosa, Mentha spicata var. cripsa ‘Moroccan’, Mentha sachalinensis, Agastache rugose, Mentha Persian, Mentha spicata ‘Russian’, Mentha requienii, Mentha arvensis, Mentha arvensis L. var. canandensis, Mentha canadensis, Mentha niliaca, Calamintha nepeta, Mentha australis, Mentha piperita ‘Canyon’, Mentha ‘wild berries’, Mentha ‘sweet pear’, Mentha laxiflora, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’, or a combination thereof.

In various embodiments of the present disclosure, each of the first and second botanicals, or constituent, derivative or extract thereof, is selected from the group consisting of: Mentha x piperita, Mentha viridis, Mentha spicata, Mentha balsamea, Mentha haplocalyx, Mentha suaveolens, Mentha suaveolens Variegata, Melissa officinalis, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata, Monarda didyma, Mentha x piperita f. citrata ‘Chocolate’, Mentha x piperita ‘Lavender’, Mentha x gracilis, Mentha x gracilis ‘Variegata’, Mentha arvensis ‘Banana’, Mentha aquatica, Mentha x villosa, Mentha nemorosa, Mentha spicata var. cripsa ‘Moroccan’, Mentha sachalinensis, Agastache rugose, Mentha Persian, Mentha spicata ‘Russian’, Mentha requienii, Mentha arvensis, Mentha arvensis L. var. canandensis, Mentha canadensis, Mentha niliaca, Calamintha nepeta, Mentha australis, Mentha piperita ‘Canyon’, Mentha ‘wild berries’, Mentha ‘sweet pear’, Mentha laxiflora, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’, or a combination thereof.

For example, at least one of the first and second botanicals, or constituent, derivative or extract thereof, may be selected from one or more (e.g. one) of the following groups:
Mentha aquatica, Mentha x piperita f. citrata ‘Lemon’,
Mentha x piperita ‘Lavender’, Mentha spicata, Mentha suaveolens ‘Variegata’,
Mentha x piperita f. citrata ‘Grapefruit’, Mentha x gracilis ‘Variegata’,
Mentha x piperita, Mentha x villosa,
or a combination thereof.
Mentha x piperita, Mentha suaveolens, Mentha x piperita f. citrata ‘Orange’, Mentha x piperita f. citrata ‘Lime’, Mentha spicata,
Mentha spicata subsp. citrata ‘Strawberry’,
or a combination thereof.
Mentha australis, Mentha x piperita f. citrata ‘Orange’, Mentha x piperita ‘Canyon’,
Mentha suaveolens ‘Variegata’, Mentha x piperita f. citrata ‘Basil’,
Mentha spicata, Mentha x piperita f. citrata ‘Chocolate’, Mentha ‘wild berries’,
or a combination thereof.
Mentha spicata var. crispa ‘Moroccan’, Mentha suaveolens, Mentha Persian, Mentha x piperita f. citrata ‘Lemon’, Mentha x gracilis ‘Variegata’,
Mentha x villosa, Mentha x piperita f. citrata ‘Lime’,
Mentha suaveolens ‘Variegata’,
or a combination thereof.
Mentha x piperita, Mentha x piperita f. citrata ‘Chocolate’, Mentha ‘sweet pear’,
Mentha spicata, Mentha arvensis ‘Banana’, Mentha x piperita citrata,
Mentha ‘wild berries’, Mentha x piperita f. citrata ‘Lemon’,
Mentha x piperita f. citrata ‘Chocolate’,
or a combination thereof.
Mentha x piperita, Mentha x piperita f. citrata ‘Grapefruit’,
Mentha x piperita f. citrata Mentha spicata,
Mentha x piperita f. citrata ‘Lemon’, Mentha spicata subsp. citrata ‘Strawberry’,
Mentha suaveolens ‘Variegate’, Mentha x piperita citrata, Mentha ‘sweet pear’,
Mentha x piperita ‘Lavender’,
or a combination thereof.

In various embodiments of the present disclosure, each of the first and second botanicals, or constituent, derivative or extract thereof is selected from one or more (e.g. one) of the above groups.

In various embodiments of the present disclosure, the first botanical or constituent, derivative or extract thereof, is a Mentha cultivar, Mentha x gracilis, Mentha arvensis, Mentha requienii or Mentha suaveolens. Examples or suitable Mentha cultivars include: Mentha x piperita f. citrata ‘Orange’, Mentha x piperita f. citrata Mentha spicata subsp. citrata ‘Strawberry’, Mentha suaveolens ‘Variegate’, Mentha x piperita f. citrata ‘Basil’, Mentha x piperita f. citrata ‘Chocolate’, Mentha ‘wild berries’, Mentha x piperita f. citrata ‘Lemon’, Mentha x gracilis ‘Variegate’, Mentha ‘sweet pear’, Mentha arvensis ‘Banana’, Mentha x piperita citrata, Mentha x piperita f. citrata ‘Grapefruit’, Mentha x piperita ‘Lavender’, or a combination thereof.

In various embodiments of the present disclosure, the second botanical or constituent, derivative or extract thereof, is selected from the group consisting of: Mentha aquatica, Mentha spicata, Mentha x piperita, Mentha x piperita f. citrata, Mentha australis, Mentha Persian, Mentha x villosa, cultivars thereof, or a mixture thereof.

In various embodiments of the present disclosure, the second botanical or constituent, derivative or extract thereof is selected from the group consisting of: Mentha aquatica, Mentha spicata, Mentha x piperita, Mentha australis, Mentha x piperita ‘Canyon’, Mentha spicata var. crispa ‘Moroccan’, Mentha Persian, Mentha x villosa, Mentha x piperita f. citrata ‘Grapefruit’, or a mixture thereof.

In various embodiments of the present disclosure, the second botanical or constituent, derivative or extract thereof is selected from the group consisting of: Mentha suaveolens, Mentha requienii, Mentha arvensis, Mentha x piperita f. citrata ‘Orange’, Mentha x piperita f. citrata Mentha spicata subsp. citrata ‘Strawberry’, Mentha suaveolens ‘Variegate’, Mentha x piperita f. citrata ‘Basil’, Mentha x piperita f. citrata ‘Chocolate’, Mentha ‘wild berries’, Mentha x piperita f. citrata ‘Lemon’, Mentha x gracilis ‘Variegate’, Mentha ‘sweet pear’, Mentha x gracilis, Mentha arvensis ‘Banana’, Mentha x piperita citrata, Mentha x piperita f. citrata ‘Grapefruit’, Mentha x piperita ‘Lavender’, or a mixture thereof.

In various embodiments of the present disclosure, at least one of the first and second botanicals, or constituent, derivative or extract thereof, is a Mentha cultivar. Suitable Mentha cultivars have already been discussed and include: Mentha x piperita f. citrata ‘Orange’, Mentha x piperita f. citrata Mentha spicata subsp. citrata ‘Strawberry’, Mentha suaveolens ‘Variegata’, Mentha x piperita f. citrata ‘Basil’, Mentha x piperita f. citrata ‘Chocolate’, Mentha ‘wild berries’, Mentha x piperita f. citrata ‘Lemon’, Mentha x gracilis ‘Variegata’, Mentha ‘sweet pear’, Mentha arvensis ‘Banana’, Mentha x piperita citrata, Mentha x piperita f. citrata ‘Grapefruit’, Mentha x piperita ‘Lavender’, or a mixture thereof.

As explained above, the composition of the present disclosure has an aroma in addition to mint which is provided by the first botanical or constituent, derivative or extract thereof and/or the second botanical or constituent, derivative or extract thereof. This concept is supported by the various species, hybrids and cultivars discussed herein, and improves the flexibility of the manufacturer when looking to prepare a mint-based product for use in a substance delivery system such as an e-cigarette, tobacco-heating device, smoking article or oral product.

It will be understood by the person skilled in the art that the present disclosure is not limited to two botanicals but includes an unlimited number or further botanicals or constituents, derivatives or extracts thereof to build the desired sensorial profile for the mint-flavored composition. The further botanicals that may be included are not necessarily limited although they are in various embodiments, selected from members of the Lamiaceae, Geraniaceae, Asteraceae, Myrtaceae or Ericaceae families. The composition may, for example, include at least a third botanical or constituent, derivative or extract thereof selected from members of the Lamiaceae family, provided that the third botanical is not spearmint, peppermint or apple mint; in other words provided that the third botanical is not selected from the group consisting of: Mentha piperita, Mentha viridis, Mentha spicata, Mentha balsamea, Mentha haplocalyx and Mentha suaveolens. The further botanicals, including the third botanical, may be defined in the same manner as the first and second botanicals discussed hereinabove.

By the expression “constituent, extract or derivative” is meant a component isolated from the botanical or an active compound naturally existing in the botanical obtained synthetically. In various embodiments of the present disclosure, the component isolated from the botanical is a natural extract, for example an oil (often referred to as an essential oil) or its constituents. Such extracts may be available commercially or may be obtained by methods known in the art. The person skilled in the art will be aware of suitable methods in this respect.

In various embodiments of the present disclosure, the component isolated from the botanical is a synthetic derivative; that's to say it is an active compound naturally existing in the botanical obtained synthetically. The compound may be commercially available or obtained by synthetic methods known in the art; again, the person skilled in the art will be aware of suitable methods in this respect.

The source of the constituent, extract or derivative for each of the botanicals is not limited in the present disclosure. The composition may be prepared from natural extracts/oils isolated from the botanicals, active compounds naturally occurring in the botanicals but obtained synthetically or a mixture thereof. In various embodiments of the present disclosure, at least one of the botanicals is a natural extract. In various embodiments of the present disclosure, each of the first and second botanicals is a natural extract. In various embodiments of the present disclosure, at least one of the botanicals is a synthetic derivative. In various embodiments, each of the first and second botanicals is a synthetic derivative.

The respective amounts of the botanicals, or constituent, derivative or extract thereof, is not limited, provided that the composition includes a level of the first botanical, or constituent, derivative or extract thereof, and a level of the second botanical, or constituent, derivative or extract thereof. As an example, each of the first and second botanicals or constituent, derivative or extract thereof may be present at an amount of at least 0.1 wt % based on the weight of the consumable as a whole. The total weight of botanicals may, for example, be an amount of up to about 85% w/w based on the total weight of the consumable. In some embodiments, the total amount of the botanicals present in the consumable is up to about 80% w/w. In some embodiments, the total amount of the botanicals present in the consumable is up to about 75% w/w, for example up to about 75 to 85% w/w. In some embodiments, the total amount of the botanicals present in the consumable is up to about 70% w/w. In some embodiments, the total amount of the botanicals present in the consumable is up to about 60% w/w. In some embodiments, the total amount of the botanicals present in the consumable is up to about 50% w/w. For example the total amount of the botanicals present in the consumable may be between about 0.1% w/w and about 60% w/w.

In other embodiments, the total amount of the botanicals present in the consumable is less than about 60% w/w. For example, less than about 50, 40, 30, 20 or 10% w/w.

In some embodiments, the total amount of the botanicals present in the consumable is up to about 50% w/w. In some embodiments, the total amount of the botanicals present in the consumable is up to about 40% w/w. In some embodiments, the total amount of the botanicals present in the consumable is up to about 30% w/w. In some embodiments, the total amount of the botanicals present in the consumable is up to about 20% w/w. In some embodiments, the total amount of the botanicals present in the consumable is up to about 15% w/w. In some embodiments, the total amount of the botanicals present in the consumable is up to about 10% w/w.

In various embodiments of the present disclosure the first botanical and second botanical, or constituent, derivative, or extract thereof, are present at a defined weight ratio. The weight ratio of first botanical, or constituent, derivative or extract thereof, to second botanical, or constituent, derivative, or extract thereof, may, for example, be from 1:10 to 10:1. In some embodiments, the weight ratio is from 1:8 to 8:1. In some embodiments, the weight ratio is from 1:5 to 5:1 or 1:4 to 4:1.

In some embodiments the botanical providing the aroma in addition to mint is included at a higher amount to the other botanical, e.g. between 10:1 to greater than 1:1, or 5:1 to greater than 1:1. For example, if the first botanical, or constituent, derivative or extract thereof, provides the aroma in addition to mint, it may be present at an equivalent amount of 10 parts and the second botanical, or constituent, derivative or extract thereof, may be present at an equivalent amount of 1 part. Alternatively, the first botanical, or constituent, derivative or extract thereof may be present at an equivalent amount of 4 parts and the second botanical, or constituent, derivative or extract thereof, may be present at an equivalent amount of 1 part.

### Consumable and the Additional Components

The consumable according to the present disclosure includes at least one further component selected from aerosol-former materials, active substances, substrate materials, or a combination thereof. The consumable or composition may further comprise one or more additional flavors, one or more sensate compounds and/or one or more other functional materials. Often these components will depend on the substance delivery system in which the consumable is intended to be used.

The consumable is an article comprising, consisting essentially of or consisting of the mint-flavored composition as defined herein and the further components listed in the claims, part of all of which is intended to be consumed during use of the substance delivery system by a user. The term “substance delivery system” is intended to encompass systems that deliver at least one substance to a user. In some embodiments, the substance to be delivered may be the composition itself. In other embodiments the substance to be delivered may be a different material to the composition: either an aerosol-generating material or a material that is not intended to be aerosolised. As appropriate, either material may comprise the one or more active constituents, one or more aerosol-former materials, one or more substrate materials.

In some embodiments, the mint-flavored composition is present in an amount of from about 0.001% to about 95% by weight of the consumable. In some embodiments, the mint-flavored composition is present in an amount of from about 0.1% to about 80% by weight of the consumable, such as 1% to about 70% by weight or 10% by weight to about 60% by weight.

In other embodiments, the mint-flavored composition is present in an amount from about 0.001% to about 20% by weight of the consumable. In some embodiments, the mint-flavored composition is present in an amount of from about 0.01% to about 15% by weight of the consumable, such as from about 0.1% to about 10% by weight of the consumable, such as from about 0.5% to about 7.5% by weight of the consumable, such as from about 1% to about 5% by weight of the consumable. In some embodiments, the mint-flavored composition is present in an amount of from about 0.1% to about 5% by weight of the consumable, such as from about 0.25% to about 4.5% by weight of the consumable, such as from about 5% to about 4% by weight of the consumable, such as from about 0.75% to about 3.5% by weight of the consumable, such as from about 1% to about 3% by weight of the consumable, such as from about 1.5% to about 2.5% by weight of the consumable. In some embodiments, the mint-flavored composition is present in an amount of from about 0.001% to about 2% by weight of the consumable, such as from about 0.01% to about 1.5% by weight of the consumable, such as from about 0.05% to about 1% by weight of the consumable, such as from about 0.1% to about 0.75% by weight of the consumable, such as from about 0.1% to about 0.5% by weight of the consumable. The skilled person will appreciate that these amounts can be combined with the amounts of the other components making up the consumable, e.g. the aerosol-former material, active substance and/or the substrate material.

The active substance as used herein may be a physiologically active material, which is a material intended to achieve or enhance a physiological response. The active substance may for example be selected from nutraceuticals, nootropics, and psychoactives. The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical.

In some embodiments, the active substance comprises nicotine or a salt thereof (e.g. nicotine ditartrate/nicotine bitartrate). In some embodiments, the active substance comprises caffeine, melatonin or vitamin B12. In various embodiments of the present disclosure, the mint-flavored composition comprises an active substance which is nicotine or a salt thereof. In other embodiments the mint-flavored composition is nicotine-free. A nicotine-free composition may include nicotine-free tobacco substitutes.

As noted herein, the active substance may comprise one or more constituents, derivatives or extracts of cannabis, such as one or more cannabinoids or terpenes. Cannabinoids are a class of natural or synthetic chemical compounds which act on cannabinoid receptors (i.e., CB1 and CB2) in cells that repress neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In various embodiments of the present disclosure, the active substance is a cannabinoid such as cannabidiol (CBD), tetrahydrocannabinol (THC), or pharmaceutically acceptable salts thereof. In various embodiments, the cannabidiol or tetrahydrocannabinol is synthetic.

The active substance may be present in any suitable amount depending on the desired dosage when consumed by the user. In various embodiments, the active substance may be present in an amount of about 0.01% by weight to about 10% by weight of the consumable. In some embodiments, the active substance may be present in an amount of about 0.01% to about 9% by weight of the consumable. In some embodiments, the active substance may be present in an amount of about 0.01% to about 8% by weight of the consumable. In some embodiments, the active substance may be present in an amount of about 0.01% to about 7% by weight of the consumable. In some embodiments, the active substance may be present in an amount of about 0.01% to about 6% by weight of the consumable. In some embodiments, the active substance may be present in an amount of about 0.01% to about 5% by weight of the consumable. In some embodiments, the active substance may be present in an amount of about 0.01% to about 4% by weight of the consumable. In some embodiments, the active substance may be present in an amount of about 0.01% to about 3% by weight of the consumable. In some embodiments, the active substance may be present in an amount of about 0.01% to about 2% by weight of the consumable. In some embodiments, the active substance may be present in an amount of about 0.01% to about 1% by weight of the consumable.

Throughout this disclosure, the term “by weight of the consumable” means the total weight of the consumable.

In other embodiments, for example when the active substance is nicotine or a salt thereof, the active subject may be present in an amount of no greater than 6% by weight of the consumable. Such as no greater than 5, 4, 3, 2, or 1% by weight. Each of these ranges may have a lower limit of 0.1, 0.2, 0.3, 0.4, 0.5, 0.6, 0.7 or 0.8% by weight.

In one embodiment, nicotine is present in an amount of from about 0.4 to about 6 wt % based on the total weight of the consumable. In one embodiment, nicotine is present in an amount of from about 0.8 to about 6 wt % based on the total weight of the consumable. In one embodiment, nicotine is present in an amount of from about 1 to about 6 wt % based on the total weight of the consumable. In one embodiment, nicotine is present in an amount of from about 1.8 to about 6 wt % based on the total weight of the consumable.

In another embodiment, nicotine is present in an amount of no greater than about 3 wt % based on the total weight of the consumable. In one embodiment, nicotine is present in an amount of from about 0.4 to about 3 wt % based on the total weight of the consumable. In one embodiment, nicotine is present in an amount of from about 0.8 to about 3 wt % based on the total weight of the consumable. In one embodiment, nicotine is present in an amount of from about 1 to about 3 wt % based on the total weight of the consumable. In one embodiment, nicotine is present in an amount of from about 1.8 to about 3 wt % based on the total weight of the consumable.

In one embodiment, nicotine is present in an amount of less than about 1.9 wt % based on the total weight of the consumable. In one embodiment, nicotine is present in an amount of less than about 1.8 wt % based on the total weight of the consumable. In one embodiment, nicotine is present in an amount of from about 0.4 to less than about 1.9 wt % based on the total weight of the consumable. In one embodiment, nicotine is present in an amount of from about 0.4 to less than about 1.8 wt % based on the total weight of the consumable. In one embodiment, nicotine is present in an amount of from about 0.5 to less than about 1.9 wt % based on the total weight of the consumable. In one embodiment, nicotine is present in an amount of from about 0.5 to less than about 1.8 wt % based on the total weight of the consumable. In one embodiment, nicotine is present in an amount of from about 0.8 to less than about 1.9 wt % based on the total weight of the consumable. In one embodiment, nicotine is present in an amount of from about 0.8 to less than about 1.8 wt % based on the total weight of the consumable. In one embodiment, nicotine is present in an amount of from about 1 to less than about 1.9 wt % based on the total weight of the consumable. In one embodiment, nicotine is present in an amount of from about 1 to less than about 1.8 wt % based on the total weight of the consumable.

In one embodiment, the consumable does not contain nicotine.

In various embodiments of the present disclosure, the consumable comprises one or more aerosol-former materials. The aerosol-former material is a material that is capable of generating aerosol, for example, when heated, irradiated or energized in any other way. The term “aerosol-generating material” may be used interchangeably with “aerosol-former material”. Aerosol-former material may, for example, be in the form of a solid, liquid or gel, which may or may not contain an active substance or ingredient as defined hereinabove. The aerosol-former material may further comprise one or more additional flavoring agents, and optionally one or more other functional material, all as defined herein.

In some embodiments, the aerosol-former material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-former material may for example comprise from about 50 wt %, 60 wt %, or 70 wt % of amorphous solid, to about wt %, 95 wt %, or 100 wt % of amorphous solid.

In some embodiments, the aerosol-former material may comprise one or more of glycerol, propylene glycol, diethylene glycol, triethylene glycol, tetraethylene glycol, 1,3-butylene glycol, erythritol, meso-erythritol, ethyl vanillate, ethyl laurate, a diethyl suberate, triethyl citrate, triethylene glycol diacetate, triacetin, a diacetin mixture, benzyl benzoate, benzyl phenyl acetate, tributyrin, lauryl acetate, lauric acid, myristic acid, and propylene carbonate. In some embodiments, the aerosol-former material comprises propylene glycol. In some embodiments, the aerosol-former material comprises glycerol, for example propylene glycol, glycerol or a mixture thereof.

In one embodiment, the aerosol-former material is present in an amount of from 10% w/w to 95% w/w based on the total weight of the consumable. In one embodiment, aerosol-former material is present in an amount of from 20% w/w to 95% w/w based on the total weight of the consumable. In one embodiment, aerosol-former material is present in an amount of from 30% w/w to 95% w/w based on the total weight of the consumable. In one embodiment, aerosol-former material is present in an amount of from 40% w/w to 95% w/w based on the total weight of the consumable.

In one embodiment, aerosol-former material is present in an amount of from 50% w/w to 90% w/w based on the total weight of the consumable. In one embodiment, aerosol-former material is present in an amount of from 50% w/w to 85% w/w based on the total weight of the consumable. In one embodiment, aerosol-former material is present in an amount of from 50% w/w to 80% w/w based on the total weight of the consumable. In one embodiment, aerosol-former material is present in an amount of from 50% w/w to 75% w/w based on the total weight of the consumable.

In one embodiment, aerosol-former material is present in an amount of at least 10% w/w based on the total weight of the consumable. In one embodiment, aerosol-former material is present in an amount of at least 20% w/w based on the total weight of the consumable. In one embodiment, aerosol-former material is present in an amount of at least 30% w/w based on the total weight of the consumable. In one embodiment, aerosol-former material is present in an amount of at least 40% w/w based on the total weight of the consumable. In one embodiment, aerosol-former material is present in an amount of at least 50% w/w based on the total weight of the consumable. In one embodiment, aerosol-former material is present in an amount of at least 55% w/w based on the total weight of the consumable. In one embodiment, aerosol-former material is present in an amount of at least 60% w/w based on the total weight of the consumable. In one embodiment, aerosol-former material is present in an amount of at least 65% w/w based on the total weight of the consumable. In one embodiment, aerosol-former material is present in an amount of at least 70% w/w based on the total weight of the consumable. In one embodiment, aerosol-former material is present in an amount of at least 75% w/w based on the total weight of the consumable. In one embodiment, aerosol-former material is present in an amount of at least 80% w/w based on the total weight of the consumable. In one embodiment, aerosol-former material is present in an amount of at least 85% w/w based on the total weight of the consumable. In one embodiment, aerosol-former material is present in an amount of at least 90% w/w based on the total weight of the consumable.

In some embodiments, the consumable comprises less than 12% w/w water. In some embodiments, the consumable comprises less than 11% w/w water. In some embodiments, consumable comprises less than 10% w/w water. In some embodiments, the consumable comprises less than 5% w/w water. In some embodiments, the consumable comprises less than 1% w/w water. In some embodiments, the consumable comprises less than 0.5% w/w water. In some embodiments, the consumable comprises substantially no water.

In one embodiment, both glycerol and propylene glycol are present as aerosol-former materials in the consumable. For example, glycerol and propylene glycol may be present in the consumable in the following amounts: 60 to 90% w/w propylene glycol; and 40 to 10% w/w glycerol, based on the total weight of glycerol and propylene glycol present in the material. In one embodiment, glycerol and propylene glycol are present in the consumable in the following amounts: 70 to 80% w/w propylene glycol, and 30 to 20% w/w glycerol, based on the total weight of glycerol and propylene glycol present in the consumable.

In one embodiment, the consumable is a liquid at about 25° C.

In various embodiments of the present disclosure, the composition optionally includes one or more sensate compounds but is free from any other additional flavorants or flavoring agents. The composition does not, for example, include a flavoring agent to provide the additional aromas mentioned above: citrus, fruity, herbal, floral, creamy, roasted, nutty or a mixture thereof.

In other embodiments of the present disclosure the composition comprises one or more additional flavors or flavoring agents which may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the additional flavor comprises menthol, spearmint and/or peppermint. In some embodiments, the additional flavor comprises eugenol. In some embodiments, the additional flavor comprises flavor components extracted from tobacco. In some embodiments, the additional flavor comprises flavor components extracted from cannabis.

In some embodiments, the total amount of the additional flavor present in the composition is up to about 10 w/w %. In some embodiments, the total amount of the additional flavor present in the composition is up to about 9 w/w %. In some embodiments, the total amount of the additional flavor present in the composition is up to about 8 w/w %. In some embodiments, the total amount of the additional flavor present in the composition is up to about 7 w/w %. In some embodiments, the total amount of the additional flavor present in the composition is up to about 6 w/w %. In some embodiments, the total amount of the additional flavor present in the composition is up to about 5 w/w %. In some embodiments, the total amount of the additional flavor present in the composition is up to about 4 w/w %. In some embodiments, the total amount of the additional flavor present in the composition is up to about 3 w/w %. In some embodiments, the total amount of the additional flavor present in the composition is up to about 2 w/w %. In some embodiments, the total amount of the additional flavor present in the composition is up to about 1 w/w %.

In various embodiments of the present disclosure, an additional flavor is present in an amount which is less than each of the first botanical or constituent, derivative or extract thereof and the second botanical or constituent, derivative or extract thereof. The additional flavor may be menthol, spearmint and/or peppermint and may be present in an amount of up to about 1 w/w %.

In various embodiments of the present disclosure, the composition further comprises one or more sensate compounds. A sensate compound is one which is intended to achieve a somatosensorial sensation usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether, and a suitable cooling agent may be, but not limited to eugenol, WS-3, WS-23. The composition may, for example, include a sensate compound selected from the group consisting of: WS-3, WS-23, carvacrol, eugenol, 1,8-cineole, and 4-methoxycinnamaldehyde.

In some embodiments, the total amount of the sensate compound present in the composition is up to about 10 w/w %. In some embodiments, the total amount of the sensate compound present in the composition is up to about 9 w/w %. In some embodiments, the total amount of the sensate compound present in the composition is up to about 8 w/w %. In some embodiments, the total amount of the sensate compound present in the composition is up to about 7 w/w %. In some embodiments, the total amount of the sensate compound present in the composition is up to about 6 w/w %. In some embodiments, the total amount of the sensate compound present in the composition is up to about 5 w/w %. In some embodiments, the total amount of the sensate compound present in the composition is up to about 4 w/w %. In some embodiments, the total amount of the sensate compound present in the composition is up to about 3 w/w %. In some embodiments, the total amount of the sensate compound present in the composition is up to about 2 w/w %. In some embodiments, the total amount of the sensate compound present in the composition is up to about 1 w/w %.

In various embodiments of the present disclosure the consumable comprises one or more substrate materials. By the term “substrate material” is meant a substance or material which supports or otherwise carries the mint-flavored composition. The substrate material may be a solid, liquid or gel. In various embodiments of the present disclosure, the substrate material is a solid or a gel. The substrate material may for example, be a fibrous material, such as a material selected from the group consisting of paper, tobacco, non-tobacco plant material (e.g. cellulose) or combinations thereof. A cellulose substrate may, for example, comprise cellulose acetate or derivatives thereof including a cellulose acetate filter in a smoking article.

The substrate may comprise a non-tobacco plant material including cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. In some embodiments, the non-tobacco plant material substrate comprises a starch, a cellulose material, or both. A cellulose material may, for example, be used to enhance certain organoleptic properties such as texture and mouthfeel, enhancing cohesiveness or compressibility of the consumable for oral use.

In some embodiments, the cellulose material may be microcrystalline cellulose (mcc). The mcc may be synthetic or semi-synthetic, or it may be obtained entirely from natural celluloses. The cellulose derivative may, for example, be present from about 10 to 80% by weight of the consumable. In some embodiments, the cellulose substrate, e.g. the cellulose derivative substrate, is present from about 10 to 70% by weight of the consumable. When a cellulose substrate is present, the consumable may comprise no more than about 10% by weight of a tobacco material, excluding any nicotine present, based on the total weight of the consumable. In various embodiments the consumable comprising a non-tobacco plant material substrate (e.g. cellulose) includes no more than about 5% by weight of a tobacco material, excluding any nicotine present, based on the total weight of the consumable.

The tobacco material, whether included in addition to the non-plant material substrate or used as a substrate itself, may be prepared from any type or form of tobacco. The present disclosure is not limited in this respect. Generally, the tobacco material is obtained from a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii.

Various representative other types of plants from the Nicotiana species are set forth in Goodspeed, The Genus Nicotiana, (Chonica Botanica) (1954); U.S. Pat. No. 4,660,577 to Sensabaugh, Jr. et al.; U.S. Pat. No. 5,387,416 to White et al., U.S. Pat. No. 7,025,066 to Lawson et al.; U.S. Pat. No. 7,798,153 to Lawrence, Jr. and U.S. Pat. No. 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Various parts or portions of the plant of the Nicotiana species can be included within a tobacco material as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment.

When nicotine is present in the consumable, it may be added or may be inherently present if the substrate material is a tobacco substrate material. In one embodiment the substrate material includes at least one tobacco substrate material. The tobacco substrate material may be solid, liquid or gel. In one embodiment, the tobacco substrate material is solid. The identity of the tobacco is not limited; it can be any type or grade of tobacco and includes any part, such as for example, the leaves or stems of any member of the genus Nicotiana and reconstituted materials thereof. In one embodiment, the tobacco is from the species Nicotiana tabacum. The tobacco substrate material may be from one variety of tobacco or from more than one variety of tobacco. As is known in the art, the latter can be referred to as a blend. Examples of tobacco varieties which may be used include, but are not limited to, Virginia, Burley, Oriental and Rustica tobaccos.

In one embodiment the tobacco substrate material is a pH-treated tobacco material; pH treatment of tobacco is well known in the art. In general, pH treatment raises the pH of the tobacco material from an acidic pH to an alkaline pH. The tobacco substrate material, including when the tobacco substrate material is a pH-treated tobacco material, can be in any suitable form. In one embodiment, the tobacco substrate material is in the form of particles, beads, granules or the like. The shape and/or size of such particles, beads or granules is not limited in the context of the present invention. The skilled person will be aware of suitable sizes and shapes and the methods by which such sizes and shapes can be achieved.

In another embodiment, the substrate material includes a non-tobacco substrate material.

The one or more other functional materials may depend on the type of substance delivery system in which the composition is to be used and may comprise one or more of pH regulators, humectants, colouring agents, preservatives, binders, fillers, stabilizers, and/or antioxidants. In particular, the pH regulator may include one or more acids selected from organic or inorganic acids. An example of an inorganic acid is phosphoric acid. The organic acid may include a carboxylic acid. The carboxylic acid may be any suitable carboxylic acid. In one embodiment, the acid is a mono-carboxylic acid. In one embodiment, the acid may be selected from the group consisting of acetic acid, lactic acid, formic acid, citric acid, benzoic acid, pyruvic acid, levulinic acid, succinic acid, tartaric acid, oleic acid, sorbic acid, propionic acid, phenylacetic acid, and mixtures thereof.

In certain embodiments, one or more humectants may be employed in the consumable, for example, with a tobacco or non-plant material substrate. Examples of humectants include, but are not limited to, glycerin, propylene glycol, and the like. Where included, the humectant is typically provided in an amount sufficient to provide desired moisture attributes to the mixture. Further, in some instances, the humectant may impart desirable flow characteristics to the mixture for depositing in a mold. When present, a humectant will typically make up about 5% or less of the weight of the mixture (e.g., from about 0.5 to about 5%). When present, a representative amount of humectant is about 0.1% to about 1% by weight, or about 1% to about 5% by weight, based on the total weight of the mixture.

### Container

The present disclosure further relates to a container or package containing the consumable as defined herein. The container or package may independently comprise one or more additional components as defined above. Specifically the container may comprise active constituents, one or more additional flavors, one or more sensate compounds, one or more aerosol-former materials, and/or one or more other functional materials.

In various embodiments of the present disclosure, the container or package is a pouched oral product comprising a saliva permeable pouch and the consumable defined herein. The consumable may be incorporated within the pouch. In such embodiments, the consumable comprises a substrate material which is a non-tobacco plant material, such as a cellulose material or a tobacco substrate material. In other embodiments of the present disclosure, the container or package is a cartomizer for an aerosol provision system and the consumable includes an aerosol-former material.

### Substance Delivery System

The present disclosure provides a substance delivery system comprising the consumable defined herein or the container or package defined herein. The substance delivery system can be implemented as a combustible aerosol provision system, a non-combustible aerosol provision system or an aerosol-free delivery system. In more detail, these systems are as follows:
combustible aerosol provision systems, such as cigarettes, cigarillos, cigars, and tobacco for pipes or for roll-your-own or for make-your-own cigarettes (whether based on tobacco, tobacco derivatives, expanded tobacco, reconstituted tobacco, tobacco substitutes or other smokeable material);
non-combustible aerosol provision systems that release compounds from an aerosol-generating material without combusting the aerosol-generating material, such as electronic cigarettes, tobacco heating products, and hybrid systems to generate aerosol using a combination of aerosol-generating materials; and
aerosol-free delivery systems that deliver the at least one substance to a user orally, nasally, transdermally or in another way without forming an aerosol, including but not limited to, lozenges, gums, patches, articles comprising inhalable powders, and oral products such as oral tobacco which includes snus or moist snuff, wherein the at least one substance may or may not comprise nicotine.

According to the present disclosure, a “combustible” aerosol provision system is one where a constituent aerosol-generating material of the aerosol provision system (or component thereof) is combusted or burned during use in order to facilitate delivery of at least one substance to a user. In some embodiments, the delivery system is a combustible aerosol provision system, such as a system selected from the group consisting of a cigarette, a cigarillo and a cigar. In some embodiments, the disclosure relates to a component for use in a combustible aerosol provision system, such as a filter, a filter rod, a filter segment, a tobacco rod, a spill, an aerosol-modifying agent release component such as a capsule, a thread, or a bead, or a paper such as a plug wrap, a tipping paper or a cigarette paper.

In various embodiments of the present disclosure, the substance delivery system is a combustible aerosol provision system. In various embodiments of the present disclosure the substance delivery system is a component of a combustible aerosol provision system such as a filter, a filter rod, a filter segment, a tobacco rod, a spill, an aerosol-modifying agent release component such as a capsule, a thread, or a bead, or a paper such as a plug wrap, a tipping paper or a cigarette paper.

According to the present disclosure, a “non-combustible” aerosol provision system is one where a constituent aerosol-generating material of the aerosol provision system (or component thereof) is not combusted or burned in order to facilitate delivery of at least one substance to a user. In some embodiments, the delivery system is a non-combustible aerosol provision system, such as a powered non-combustible aerosol provision system. In some embodiments, the non-combustible aerosol provision system is an electronic cigarette, also known as a vaping device or electronic nicotine delivery system (END), although it is noted that the presence of nicotine in the aerosol-generating material is not a requirement. In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

In various embodiments of the present disclosure, the substance delivery system is a non-combustible aerosol provision system. Typically, the non-combustible aerosol provision system may comprise a non-combustible aerosol provision device and a consumable for use with the non-combustible aerosol provision device. In some embodiments, the disclosure relates to consumables comprising aerosol-former material and the mint-flavored composition, and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate, which may be energized to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent. In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

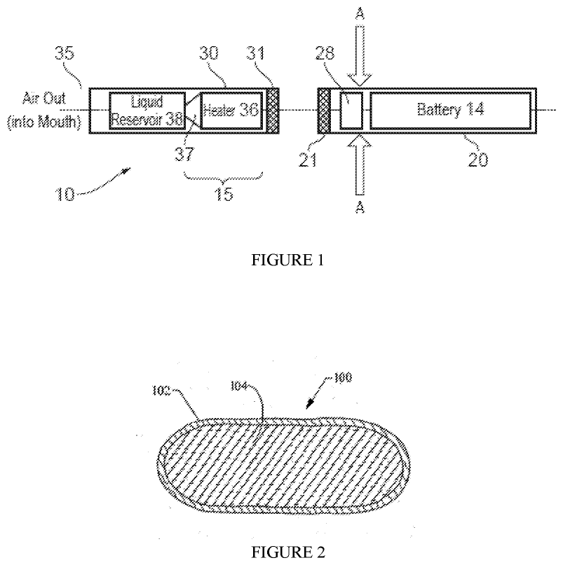

FIG. 1 is a highly schematic diagram (not to scale) of an example aerosol provision system, such as an e-cigarette 10, to which embodiments are applicable. The e-cigarette has a generally cylindrical shape, extending along a longitudinal axis indicated by a dashed line (although aspects of the present disclosure are applicable to e-cigarettes configured in other shapes and arrangements), and comprises two main components, namely an aerosol provision device 20 and an article 30.

The article 30 includes a store for aerosolizable material (source liquid) 38 containing an aeroslizable material (source liquid) from which an aerosol is to be generated. This material may correspond to the consumable according to the present disclosure or the consumable may be included in a separate compartment through which heated air passes through (not shown in FIG. 1). The article 30 further comprises an aerosol generating component (heating element or heater) 36 for heating aerosolizable material to generate the aerosol. A transport element or wicking element or wick 37 is provided to deliver aersolizable material from the store 38 to the heating element 36. A part or parts of the wick 37 are in fluid communication with aerosolizable material in the store 38 and by a wicking or capillary action aerosolizable material is drawn along or through the wick 37 to a part or parts of the wick 37 which are in contact with the heater 36.

Vaporization of the aerosolizable material occurs at the interface between the wick 37 and the heater 36 by the provision of heat energy to the aerosolizable material to cause evaporation, thus generating the aerosol. The aerosolizable material, the wick 37 and the heater 36 may be collectively referred to as an aerosol or vapor source. The wick 37 and the heater 36 may be collectively referred to as a vaporizer or an atomiser 15. Typically, a single wick will be present, but it is envisaged that more than one wick could be present, for example, two, three, four or five wicks. The wick may be formed a sintered material. The sintered material may comprise sintered ceramic, sintered metal fibers/powders, or a combination of the two. The (or at least one of/all of the) sintered wick(s) may have deposited thereon/embedded therein an electrically resistive heater. Such a heater may be formed from heat conducting alloys such as NiCr alloys. Alternatively, the sintered material may have such electrical properties such that when a current is passed there through, it is heated. Thus, the aerosol-generating component and the wick may be considered to be integrated. In some embodiments, the aerosol-generating component and the wick are formed from the same material and form a single component.

The article 30 further includes a mouthpiece 35 having an opening through which a user may inhale the aerosol generated by the vaporizer 15. The aerosol for inhalation may be described as an aerosol stream or inhalable airstream.

The aerosol delivery device 20 includes a power source (a re-chargeable cell or battery 14, referred to herein after as a battery) to provide power for the e-cigarette 10, and a controller (printed circuit board (PCB)) 28 and/or other electronics for generally controlling the e-cigarette 10. The aerosol delivery device can therefore also be considered as a battery section, or a control unit or section. During operation of the device, the controller will determine that a user has initiated a request for the generation of an aerosol. This could be done via a button on the device which sends a signal to the controller that the aerosol generator should be powered. Alternatively, a sensor located in or proximal to the airflow pathway could detect airflow through the airflow pathway and convey this detection to the controller. A sensor may also be present in addition to the presence of a button, as the sensor may be used to determine certain usage characteristics, such as airflow, timing of aerosol generation etc. For example, in use, when the heater 36 receives power from the battery 14, as controlled by the circuit board 28 possibly in response to pressure changes detected by an air pressure sensor (not shown), the heater 36 vaporizes aerosolizable material delivered by the wick 37 to generate the aerosol, and this aerosol stream is then inhaled by a user through the opening in the mouthpiece 35. The aerosol is carried from the aerosol source to the mouthpiece 35 along an air channel (not shown in FIG. 1) that connects the aerosol source to the mouthpiece opening as a user inhales on the mouthpiece.

In this particular example, the device 20 and article 30 are detachable from one another by separation in a direction parallel to the longitudinal axis, as shown in FIG. 1, but are joined together when the system 10 is in use by cooperating engagement elements 21, 31 (for example, a screw, magnetic or bayonet fitting) to provide mechanical and electrical connectivity between the device 20 and the article 30, in particular connecting the heater 36 to the battery 14. The battery may be charged as is known to one skilled in the art.

In some embodiments, the article comprises/forms a sealed container. For example, the sealed container may be hermetically sealed. The hermetically sealed container may comprise a blister pack with one or more hermetically sealed compartments for storage of one or more articles comprising the composition described herein.

In some embodiments, the substance delivery system is an aerosol-free delivery system that delivers at least one substance to a user orally, nasally, transdermally or in another way without forming an aerosol, including but not limited to, lozenges, gums, patches, articles comprising inhalable powders, and oral products such as oral tobacco which includes snus or moist snuff, wherein the at least one substance may or may not comprise nicotine.

In various embodiments of the present disclosure, the substance delivery system is an aerosol-free delivery system. The aerosol-free delivery system may, for example, be a smokeless tobacco composition comprising the consumable where at least one tobacco material acts as a substrate for the mint-flavored composition, or an oral product where the oral product is in the form of a pouch comprising the consumable. FIG. 2 is a cross-sectional view of a pouched product 100, taken across the width of the product, showing an outer pouch 102 filled with a consumable 104 of the present disclosure.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilized and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future.

1. A consumable for use in a substance delivery system, wherein the consumable comprises a mint-flavored composition comprising:
(i) a first botanical or constituent, derivative or extract thereof;
(ii) a second, different, botanical or constituent, derivative or extract thereof;
wherein the first and second botanicals are selected from members of the Lamiaceae, Geraniaceae, Asteraceae, Myrtaceae or Ericaceae families;
provided that if the first botanical is spearmint or peppermint selected from the group consisting of Mentha x piperita, Mentha viridis, Mentha spicata, Mentha balsamea and Mentha haplocalyx, the second botanical is not spearmint or peppermint selected from the group consisting of Mentha x piperita, Mentha viridis, Mentha spicata, Mentha balsamea and Mentha haplocalyx;
wherein the composition has an aroma in addition to mint which is provided by (i) and/or (ii), wherein at least one of the botanicals is a synthetic derivative, and the consumable further comprises at least one component selected from aerosol-former materials, active substances, substrate materials, or a combination thereof.

2. The consumable of claim 1, wherein at least one of the first and second botanicals, or constituent, derivative or extract thereof, is a member of the Lamiaceae family.

3. The consumable of claim 2, wherein at least one of the first and second botanicals, or constituent, derivative or extract thereof, is a member of the Menthae tribe.

4. The consumable of claim 3, wherein at least one of the first and second botanicals, or constituent, derivative or extract thereof, is a member of Mentha genus.

5. The consumable of claim 1, wherein at least one of the first and second botanicals, or constituent, derivative or extract thereof, is a member of the Ericaceae family, such as a member of the Gaultheria genus.

6. The consumable of claim 1, wherein at least one of the first and second botanicals, or constituent, derivative or extract thereof, is a member of the Eucalyptus genus.

7. The consumable of claim 1, wherein at least one of the first and second botanicals, or constituent, derivative or extract thereof, is a member of the Geraniaceae, Asteraceae, Myrtaceae, Ericaceae or Lamiaceae families but is not a member of Mentha genus.

8. The consumable of claim 7, wherein at least one of the first and second botanicals, or constituent, derivative or extract thereof is selected from the group consisting of: Agastache rugose, Calamintha nepeta, Tagetes lemmonii, Perlargonium ‘Pungent Peppermint’, Pelargonium x scarboroviae, Pycnanthemum pilosum, Micromeria fruticose, Eucalyptus radiata, Agastache rupestris ‘Apache Sunset’, Salvia elegans, Salvia elegans ‘Tangerine’, Salvia dorisiana, Salvia elegans ‘Honey Melon’, Salvia discolor, Salvia melissodora, Ocimum basilicum ‘cinnamon’, Ocimum basilicum ‘anise’ Ocimum selloi, Thymus sp., Thymus ‘TM122’, Thymus sp., or hybrids and/or cultivars thereof.

9. The consumable of claim 1, wherein at least one of the first and second botanicals, or constituent, derivative or extract thereof, is selected from the group consisting of:
Mentha x piperita, Mentha viridis, Mentha spicata, Mentha balsamea,
Mentha haplocalyx, Mentha suaveolens, Mentha suaveolens Variegata,
Melissa officinalis, Mentha x piperita f citrata ‘Lemon’,
Mentha x piperita f citrata ‘Basil’, Mentha x piperita f citrata ‘Lime’,
Mentha x piperita f citrata ‘Grapefruit’, Mentha spicata subsp. citrata ‘Strawberry’,
Mentha x piperita f citrata ‘Orange’, Mentha x piperita f citrata, Monarda didyma,
Mentha x piperita f citrata ‘Chocolate’,
Mentha x piperita ‘Lavender’, Mentha x gracilis, Mentha x gracilis ‘Variegata’, Mentha arvensis ‘Banana’, Mentha aquatica, Mentha x villosa, Mentha nemorosa, Mentha spicata var. cripsa ‘Moroccan’, Mentha sachalinensis, Agastache rugose, Mentha spicata var. crispa ‘Persian’,
Mentha spicata ‘Russian’, Mentha requienii, Mentha arvensis, Mentha arvensis L. var. canadensis, Mentha canadensis, Mentha niliaca, Calamintha nepeta, Mentha australis,
Mentha piperita ‘Canyon’, Mentha ‘wild berries’,
Mentha ‘sweet pear’, Mentha laxiflora, Mentha ‘Iced hazelnut’,
Mentha ‘Berries and Cream’, Mentha ‘Pink Candypops’, Mentha ‘Marshmallow’,
Mentha ‘Oregano-Thyme’, Mentha ‘Sister Julie's Wintergreen’,
Mentha ‘Jim's Candy Lime’, Mentha ‘Margarita’, Mentha ‘Sweet Bay’,
Pycnanthemum pilosum, Micromeria fruticose,
Perlargonium ‘Pungent Peppermint’, Pelargonium x scarboroviae,
Agastache rupestris ‘Apache Sunset’, Salvia elegans, Salvia elegans ‘Tangerine’,
Eucalyptus radiata, Tagetes lemmonii, Salvia dorisiana,
Salvia elegans ‘Honey Melon’, Salvia discolor, Salvia melissodora,
Ocimum basilicum ‘cinnamon’, Ocimum basilicum ‘anise’, Ocimum selloi, Thymus sp., Thymus ‘TM122’, Thymus sp., Gaultheria procumbens, or hybrids and/or cultivars thereof.

10. The consumable of claim 1, wherein each of the first and second botanicals, or constituent, derivative or extract thereof, are independently selected from the group consisting of:
(1) Mentha aquatica, Mentha x piperita f citrata ‘Lemon’, Mentha x piperita ‘Lavender’, Mentha spicata, Mentha suaveolens ‘Variegata’, Mentha x piperita f citrata ‘Grapefruit’, Mentha x gracilis ‘Variegata’, Mentha x piperita, Mentha x villosa, or hybrids and/or cultivars thereof;
(2) Mentha x piperita, Mentha suaveolens, Mentha x piperita f citrata ‘Orange’, Mentha x piperita f citrata ‘Lime’, Mentha spicata, Mentha spicata subsp. citrata ‘Strawberry’, or hybrids and/or cultivars thereof;
(3) Mentha australis, Mentha x piperita f citrata ‘Orange’, Mentha x piperita ‘Canyon’, Mentha suaveolens ‘Variegata’, Mentha x piperita f citrata ‘Basil’, Mentha spicata, Mentha x piperita f citrata ‘Chocolate’, Mentha ‘wild berries’, or hybrids and/or cultivars thereof;
(4) Mentha spicata var. crispa ‘Moroccan’, Mentha suaveolens, Mentha spicata var. cripsa ‘Persian’, Mentha x piperita f citrata ‘Lemon’, Mentha x gracilis ‘Variegata’, Mentha x villosa, Mentha x piperita f citrata ‘Lime’, Mentha suaveolens ‘Variegate’, or hybrids and/or cultivars thereof;
(5) Mentha x piperita, Mentha x piperita f citrata ‘Chocolate’, Mentha ‘sweet pear’, Mentha spicata, Mentha arvensis ‘Banana’, Mentha x piperita citrata, Mentha ‘wild berries’, Mentha x piperita f citrata ‘Lemon’, Mentha x piperita f citrata ‘Chocolate’, or hybrids and/or cultivars thereof; or
(6) Mentha x piperita, Mentha x piperita f citrata ‘Grapefruit’, Mentha x piperita f citrata ‘Lime’, Mentha spicata, Mentha x piperita f citrata ‘Lemon’, Mentha spicata subsp. citrata ‘Strawberry’, Mentha suaveolens ‘Variegate’, Mentha x piperita citrata, Mentha ‘sweet pear’, Mentha x piperita ‘Lavender’, or hybrids and/or cultivars thereof.

11. The consumable of claim 1, wherein the first botanical, or constituent, derivative or extract thereof, is selected from the group consisting of: Mentha suaveolens, Mentha x piperita f citrata ‘Orange’,
Mentha x piperita f citrata ‘Lime’, Mentha spicata subsp. citrata ‘Strawberry’, Mentha suaveolens ‘Variegate’, Mentha x piperita f citrata ‘Basil’,
Mentha x piperita f citrata ‘Chocolate’, Mentha ‘wild berries’,
Mentha x piperita f citrata ‘Lemon’, Mentha x gracilis ‘Variegata’,
Mentha ‘sweet pear’, Mentha x gracilis, Mentha arvensis ‘Banana’, Mentha x piperita citrata, Mentha x piperita f citrata ‘Grapefruit’, Mentha x piperita ‘Lavender’, or hybrids and/or cultivars thereof.

12. The consumable of claim 11, wherein the second botanical, or constituent, derivative or extract thereof, is selected from the group consisting of:
Mentha aquatica, Mentha spicata, Mentha x piperita, Mentha australis,
Mentha x piperita ‘Canyon’, Mentha spicata var. crispa ‘Moroccan’,
Mentha Persian, Mentha x villosa, Mentha x piperita fcitrata ‘Grapefruit’,
or hybrids and/or cultivars thereof.

13. The consumable of claim 1, wherein the composition comprises a third botanical or constituent, derivative or extract thereof, wherein the third botanical is selected from members of the Lamiaceae, Geraniaceae, Asteraceae, Myrtaceae or Ericaceae families; provided that the third botanical is not peppermint, spearmint or apple mint (Mentha suaveolens).

14. The consumable of claim 1, wherein the additional aroma is selected from the group consisting of: citrus, fruity, floral, herbal, creamy (e.g. chocolate), roasted, nutty or a combination thereof.

15. The consumable of claim 1, wherein the additional aroma is selected from the group consisting of: lemon, bergamot, orange, floral, grapefruit, lime, banana, pineapple, apple, pear, mango, basil, cinnamon, chocolate, strawberry, raspberry, ginger, lavender, coconut, melon, blackcurrant, grape, anise, rose, liquorice, root beer, coconut, grape, green pepper, hazelnut, marshmallow, peach, thyme, basil, oregano, bay leaf, or a combination thereof.

16. The consumable of claim 1, wherein the composition further comprises one or more sensate compounds.

17. The consumable of claim 1, wherein the active substance is a physiologically active substance, such as a substance selected from the group consisting of nicotine, caffeine, taurine, theine, vitamins such as B6 or B12 or C, melatonin, cannabinoids, or constituents, derivatives or combinations thereof.

18. The consumable of claim 17, wherein the active substance is nicotine.

19. The consumable of claim 1 in the form of a solid, liquid or gel.

20. The consumable of claim 1, wherein the composition is nicotine-free.

21. The consumable of claim 1, wherein at least one of the botanicals is a natural extract.

22. (canceled)

23. The consumable of claim 1, wherein the mint-flavoured composition is present in an amount of from about 0.001% to about 20% by weight of the consumable.

24. The consumable of claim 1 wherein the further component comprises an aerosol-former material selected from one or more of glycerol, propylene glycol, diethylene glycol, triethylene glycol, tetraethylene glycol, 1,3-butylene glycol, erythritol, meso-erythritol, ethyl vanillate, ethyl laurate, a diethyl suberate, triethyl citrate, triacetin, a diacetin mixture, benzyl benzoate, benzyl phenyl acetate, tributyrun, lauryl acetate, lauric acid, myristic acid, and propylene carbonate.

25. The consumable of claim 1, wherein the further component comprises a substrate material, wherein the substrate material is a fibrous material, such as a material selected from the group consisting of paper, tobacco, non-tobacco plant material (e.g. cellulose) or combinations thereof.

26. A container or package containing the consumable of claim 1.

27. The container or package of claim 26 which is a pouched oral product comprising a saliva permeable pouch.

28. A substance delivery system comprising the consumable of claim 1 or the container of claim 26.

29. The substance delivery system of claim 28, wherein the system is a combustible aerosol provision system, a non-combustible aerosol provision system or an aerosol-free delivery system.

30. A process for preparing a consumable as defined in claim 1, the process comprising:
(i) providing a first botanical or constituent, derivative or extract thereof;
(ii) providing a second, different botanical or constituent, derivative or extract thereof; wherein the first and second botanicals are selected from members of the Lamiaceae, Geraniaceae, Asteraceae, Myrtaceae or Ericaceae families; provided that if the first botanical is spearmint or peppermint, the second botanical is not spearmint or peppermint;
(iii) contacting (i) and (ii) to prepare the mint-flavored composition;
(iv) combining the mint-flavored composition with a further component selected from aerosol-former materials, active substances, substrate materials, or a combination thereof, to prepare the consumable.

31. Use of a botanical, or constituent, derivative or extract thereof, in a mint-flavored composition to provide an aroma in addition to mint, wherein the botanical, or constituent, derivative or extract thereof, is selected from members of the Geraniaceae, Asteraceae, Myrtaceae, Ericaceae, or Lamiaceae families provided that it is not a member of the Mentha genus, wherein the botanical comprises a synthetic derivative, wherein the composition is for use in a substance delivery system.

32. Use of a botanical, or constituent, derivative or extract thereof to provide a mint aroma to a user of a substance delivery system, wherein the botanical or constituent, derivative or extract thereof is a member of a genus other than Mentha, wherein the botanical comprises a synthetic derivative.

33. Use of a botanical, or constituent, derivative or extract thereof, in a consumable for a substance delivery system to provide an aroma in addition to mint, wherein the botanical, or constituent, derivative or extract thereof is selected from members of the Lamiaceae, Geraniaceae, Asteraceae, Myrtaceae or Ericaceae families, and the consumable further comprises at least one component selected from aerosol-former materials, active substances, substrate materials, or a combination thereof, wherein the botanical comprises a synthetic derivative.
